(19) United States
(12) Patent Application Publication
(10) Pub. No.: US 2005/0053632 A1
(43) Pub. Date: Mar. 10, 2005

(54) Cosmetic or dermatological preparations having a long-term cooling action

(75) Inventors: Andreas Schafer, Hamburg (DE); Andreas Bleckmann, Ahrensburg (DE); Boris Syskowski, Hamburg (DE); Rainer Kropke, Schenefeld (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/759,946

(22) Filed: Jan. 16, 2004

Related U.S. Application Data
(63) Continuation of application No. PCT/EP02/07784, filed on Jul. 12, 2002.

(30) Foreign Application Priority Data
Jul. 17, 2001 (DE) 101 34 607.7

(51) Int. Cl. A61K 7/00

(52) U.S. Cl. 424/401

(57) ABSTRACT

The invention is a cooling cosmetic or medical topical preparation, comprising one or more substances having a melting point or melting interval between 28° C. and 40° C. and a positive melting enthalpy, wherein the one or more substances are present in the preparation in the solid state.

### CROSS-REFERENCE TO RELATED APPLICATIONS

This is a continuation application of PCT/EP02/07784, filed Jul. 12, 2002, which is incorporated herein by reference in its entirety, and also claims the benefit of German Priority Application No.101 34 607.7, filed Jul. 17, 2001.

### FIELD OF INVENTION

The present invention relates to cosmetic and dermatological preparations having a long-term cooling action, in particular to skin care cosmetic and dermatological preparations.

### BACKGROUND OF THE INVENTION

The skin is the largest human organ. Amongst its many functions (for example for heat regulation and as a sensory organ), the barrier function, which prevents the skin (and thus ultimately the entire organism) from drying out, is probably the most important. At the same time, the skin acts as a protective device against the penetration and absorption of external substances. This barrier function is effected by the epidermis which, as the outermost layer, forms the actual protective sheath against the environment. Being about one tenth of the total thickness, it is also the thinnest layer of the skin.

The outermost layer of the epidermis, the stratum corneum (horny layer), is an important barrier layer and therefore of particular significance inter alia for protecting against environmental influences and drying out. As a result of contact with the environment, the horny layer is continually worn away and must therefore be continuously renewed.

A model for the skin which is widely used today in the expert field depicts the stratum corneum as a two-component system, similar to a brick wall (bricks and mortar model). In this model, the corneocytes (horny cells) are the bricks and the complex lipid membrane in the intercellular spaces is the mortar.

Apart from its barrier effect against external chemical and physical influences, the epidermal lipids also contribute to the holding together of the horny layer and influence the skin smoothness. In contrast to sebaceous gland lipids, which do not form a continuous film on the skin, the epidermal lipids are distributed over the entire horny layer.

The extremely complex interaction of the moisture-binding substances and of the lipids in the upper layers of the skin is very important for regulation of skin moisture. Cosmetics thus usually comprise water-binding substances in addition to balanced lipid mixtures and water.

As well as the chemical composition, the physical behavior of these substances is, however, also of importance. The development of highly biocompatible emulsifiers and surfactants with liquid-crystalline properties is thus desirable. Products formulated therewith support the liquid-crystalline organization of the intercellular lipids of the stratum corneum and thus improve the barrier properties of the horny layer. It is particularly advantageous if the molecular constituents of such products consist of substances which are naturally occurring in the epidermis.

The main role of cosmetic skin care is taken to be the strengthening or restoration of the skin's natural function as a barrier against environmental influences (e.g. dirt, chemicals, microorganisms) and against the loss of endogenous substances (e.g. water, natural fats, electrolytes).

Impairment of this function can lead to increased resorption of toxic or allergenic substances or to attack by microorganisms and, consequently, to toxic or allergic skin reactions.

Another aim of skin care is to replenish the fats and water lost from the skin as a result of daily washing. This is particularly important when the natural regeneration ability is inadequate. In addition, skincare products should protect against environmental influences, in particular against sun and wind, and delay skin ageing.

Medical topical compositions normally comprise one or more medicaments in an effective concentration. For the sake of simplicity, reference is made to the legal provisions of the Federal Republic of Germany (e.g. Cosmetics Regulation, Foods and Drugs Act) for a clear distinction between cosmetic and medical use and corresponding products.

Customary forms in which cosmetics are applied are emulsions, i.e. metastable two-phase or multi-phase systems in which the individual phases are in the liquid state. The most common emulsions are O/W and W/O emulsions. Less common application forms are multiple emulsions, i.e. those which in the droplets of the dispersed (or discontinuous) phase for their part comprise droplets of a further dispersed phase, e.g. W/O/W emulsions and O/W/O emulsions.

In order to be able to ensure the metastability of emulsions, interface-active substances, i.e. emulsifiers, are generally necessary.

It is possible to prepare emulsifier-free preparations which have, for example, in an aqueous phase, dispersed oil droplets, similar to an O/W emulsion. A prerequisite for this may be that the continuous aqueous phase has a gel backbone which stabilizes the dispersed phase and other conditions besides. Such systems are sometimes called hydrodispersions or oleodispersions depending on which is the disperse phase and which is the continuous phase.

Customary cosmetic and dermatological preparation forms which have become ever more widespread in recent times are gels.

In the technical sense, gels are understood as meaning: relatively dimensionally stable, readily deformable disperse systems of at least two components, which usually consist of an (in most cases solid) colloidally dispersed substance of long-chain molecule groups (e.g. gelatin, silica, polysaccharides) as structure former and a liquid dispersant (e.g. water). The colloidally dispersed substance is often referred to as a thickener or gelling agent. It forms a spatial network within the dispersant, where individual colloidal particles may be joined together with greater or lesser strength via electrostatic interaction. The dispersant which surrounds the network is characterized by electrostatic affinity to the gelling agent, i.e. a predominantly polar (in particular: hydrophilic) gelling agent preferably gels a polar dispersant (in particular: water), whereas a predominantly nonpolar gelling agent preferably gels nonpolar dispersants.

Strong electrostatic interactions which are realized, for example, in hydrogen bridge bonds between gelling agent and dispersant, but also between dispersant molecules with one another, can lead to considerable crosslinking also of the dispersant. Hydrogels can consist of virtually 100% of water (in addition, for example, to about 0.2-1.0% of a gelling agent) and have entirely solid consistency. The water content is present here in ice-like structural elements, meaning that gels therefore do justice to the origin of their name [from Latin “gelatum”=“frozen” via the alchemistic term “gelatina” (16th century) for the modern term “gelatin”].

In cosmetic and pharmaceutical technology, lipogels and oleogels (of waxes, fats and fatty oils), and also carbogels (of paraffin or petrolatum) are also common. In practice, a distinction is made between oleogels, which are virtually free from water, and hydrogels, which are virtually free from fat. In most cases, gels are transparent. In cosmetic or pharmaceutical technology, gels are usually characterized by a semisolid, often flowable consistency.

In addition, so-called surfactant gels are customary preparations of the prior art. This is understood as meaning systems which, as well as water, have a high concentration of emulsifiers, typically more than about 25% by weight, based on the overall composition. If oil components are solubilized in these surfactant gels, microemulsion gels are obtained which are also referred to as “ringing gels”. By adding nonionic emulsifiers, for example alkyl polygylcosides, it is possible to obtain cosmetically more elegant microemulsion gels.

The cooling action of cosmetic preparations has hitherto been based on two basic principles:

Use of components which evaporate in a gas-like manner following topical application and take the amount of energy required for this, the so-called enthalpy of evaporation, for the most part from the surface of the skin. Suitable liquid components are therefore used in corresponding non-occlusive cosmetics. In this connection, ethanol has proven particularly suitable, and formulations with a high water content likewise exhibit a significant cooling action.

Use of so-called cooling agents which interact with the heat receptors in the skin and thus trigger a sensation of cold without generating a measurable physical cooling. For this purpose, menthol and various menthol derivatives (Frescolate, Physcool, Questice L, etc.) are used in particular. As well as the irritative potential, in particular due to their significant intrinsic odor, particularly highly ethanol contents, and also menthol and its derivatives are unsuitable for numerous intended uses from olfactory points of view. Moreover, such substances often enough bring about an increase in circulation at the same time, which, by contrast, causes a feeling of warmth.

The literature describes, for example, ionic compounds, in particular ammonium salts, as cooling agents. Cooling preparations which are also used widely are isopropanolic gels with an addition of camphor and menthol.

The use of these substances, namely on irritated skin, however, is problematic. Moreover, many of these compounds are sparingly soluble in water. Their use is consequently limited to a few cosmetics and dermatological compositions.

### SUMMARY OF THE INVENTION

An object of the present invention was therefore to provide care cosmetic and medicinal preparations which do not have the disadvantages of the prior art, in particular those which, applied to the skin or mucous membranes, have a wetting and/or cooling action.

DE 43 12 656 describes the use of cosmetically or pharmaceutically acceptable substances with positive solution enthalpy in cosmetic or medicinal topical preparations, characterized in that the substance or the substances in the preparations are present in a largely anhydrous medium and/or are shielded from a water-containing medium by a material barrier.

Although this procedure can in principle lead to cosmetically satisfactory preparations, these are, however, extremely complex to prepare in pharmaceutical technology.

It was thus the object of the present invention to overcome the shortcomings of the prior art and to provide cooling cosmetic or dermatological preparations which are easy to produce, do not exert an irritative effect on skin or mucous membranes, and afford pleasant cooling when applied in accordance with directions.

For numerous compounds the phase transition from the solid state to the liquid state is likewise characterized by the occurrence of heat transformation. Endothermic melting processes take place with the absorption of energy which comes from the surrounding area where it thus leads to a temperature drop.

It was, however, surprising and could not have been foreseen by the person skilled in the art that cooling cosmetic or medicinal topical preparations characterized by
a content of one or more substances,
   whose melting point or melting interval is chosen in the range between 28° C. and 40° C., and which
   have positive melting enthalpy,
where this substance or substances are present in the preparations in the solid state.

In addition, the invention relates to the use of
one or more substances,
   whose melting point or melting interval is chosen in the range between 28° C. and 40° C., and which
   have positive melting enthalpy for producing cosmetic or medicinal topical preparations with a cooling action,
where this substance or substances are present in the preparations in the solid state.

### DETAILED DESCRIPTION OF THE PREFERRED EMBODIMENTS

These preparations according to the invention develop their cooling action within a broad use concentration range of the underlying substances, for example 0.5-50% by weight, advantageously 1-20% by weight of these substances, based on the total weight of the preparations. They are easy to formulate and pose no great requirements on manufacturing procedures.

If, as is particularly advantageous according to the invention, lipophilic substances are chosen whose melting point or melting interval is in the range between 28° C. and 40° C., and which have positive melting enthalpy, then particularly markedly hydrophilic cosmetic or dermatological bases therefor are suitable in which the substance or as the substances whose melting point or melting interval is in the range between 28° C. and 40° C., and which have positive melting enthalpy, are then particularly preferably present in a finely particulate suspension, for example in aqueous gel formulations.

Particularly advantageous substances whose melting point or melting interval is chosen in the range between 28° C. and 40° C., and which have positive melting enthalpy are chosen from the group of lipophilic substances, in particular:
the esters of C₁₄-C₁₈ fatty acids, with low molecular weight (C₁-C₅) alcohols,
the fatty alcohols C₈-C₂₂,
the branched or unbranched fatty acids,
the hydrocarbons C₁₆-C₂₂.

For the purposes of the present invention, particular preference is given to methyl palmitate.

Moreover, further cooling active ingredients, for example menthol or menthol derivatives, can optionally also be added to these formulations for sensory modification or to enhance the cooling effect.

The substance or the substances whose melting point or melting interval is chosen in the range between 28° C. and 40° C. and which have positive melting enthalpy are also referred to collectively as active ingredient according to the invention, or are provided with similar synonyms, for the purposes of the present disclosure.

According to the invention, it is particularly extremely advantageous to use the active ingredient used according to the invention or cosmetic or topical dermatological preparations with an active content of active ingredient used according to the invention for the cosmetic or dermatological treatment or prophylaxis of undesired skin conditions.

According to the invention, customary antioxidants may be used preparations which comprise the active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoides, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to pmol/kg), and also (metal) chelating agents (e.g. α-hydroxy fatty acids, palmitic acid, phytic acid, lactoferrin) α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechins, vitamin C and derivatives (e.g. ascorbyl palmitate, Mg ascorbyl phosphate, ascorbyl acetate) tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations with an active content of active ingredient used according to the invention is carried out in the usual manner, by applying the active ingredient used according to the invention or the cosmetic or topical dermatological preparations with an active content of active ingredient used according to the invention to the affected areas of skin.

The active ingredient used according to the invention can advantageously be incorporated into customary cosmetic and dermatological preparations, which may be in various forms. Thus, they may, for example, be a solution, an emulsion of the water-in-oil (W/O) type or of the oil-in-water (O/W) type, or a multiple emulsions, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without the customary auxiliaries and additives. These include, for example, consistency-imparting agents, fillers, perfume, dyes, emulsifiers, additional active ingredients, such as vitamins or proteins, light protection agents, stabilizers, insect repellents, alcohol, water, salts, antimicrobicidally, proteolytically or keratolytically active substances, etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

In this connection, it is likewise advantageous to add the active ingredient used according to the invention as additive to preparations which already comprise other active ingredients for other purposes.

Accordingly, cosmetic or topical dermatological compositions for the purposes of the present invention can, depending on their formulation, be used, for example, as skin protection cream, cleansing milk, sunscreen lotion, nutrient cream, day or night cream etc. It is optionally possible and advantageous to use the compositions according to the invention as a basis for pharmaceutical formulations.

Also favorable are in some cases those cosmetic and dermatological preparations which are in the form of a sunscreen. Preferably, as well as the active ingredient used according to the invention, these additionally comprise at least one UVA filter substance and/or at least one UVB filter substance and/or at least one inorganic pigment.

It is, however, also advantageous for the purposes of the present inventions to create both cosmetic and dermatological preparations whose main purpose is not protection against sunlight but which nevertheless comprise a content of UV protection substances. Thus, UV-A and/or UV-B filter substances are usually incorporated into day creams, for example.

Preparations according to the invention can advantageously comprise substances which absorb UV radiation in the UVB region, the total amount of filter substances being, for example, 0.1% by weight to 30% by weight, preferably 0.5 to 10% by weight, in particular 1 to 6% by weight, based on the total weight of the preparations.

The UVB filters may be oil-soluble or water-soluble. Examples of oil-soluble substances are:
3-benzylidenecamphor and derivatives thereof, e.g. 3-(4-methylbenzylidene)camphor,
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzal malonate;
2,4,6-trianilino(p-carbo-2′-ethyl-1′-hexyloxy)-1,3,5-triazine.

Water-soluble substances are advantageously:
2-phenylbenzimidazole-5-sulfonic acid and salts thereof, e.g. sodium, potassium or triethanolammonium salts,
sulfonic acid derivatives of benzophenones, preferably 2-hydroxy-4-methoxybenzophenone-5-sulfonic acid and its salts;
sulfonic acid derivatives of 3-benzylidenecamphor, such as, for example 4-(2-oxo-3-bornylidenemethyl)benzenesulfonic acid, 2-methyl-5-(2-oxo-3-bornylidenemethyl)sulfonic acid and its salts.

The list of specified UVB filters which can be used according to the invention is not of course intended to be limiting.

The invention also provides the combination of a UVA filter according to the invention with a UVB filter or a cosmetic or dermatological preparation according to the invention which also comprises a UVB filter.

It may also be advantageous to use UVA filters which are customarily present in cosmetic and/or dermatological preparations in preparations according to the invention. Such filter substances are preferably derivatives of dibenzoylmethane, in particular 1-(4′-tert-butylphenyl )-3-(4′-methoxyphenyl)propane-1,3-dione and 1-phenyl-3-(4′-isopropylphenyl)propane-1,3-dione. Preparations which comprise these combinations are also provided by the invention. The amounts of UVA filter substances which may be used are the same as have been mentioned for UVB filter substances.

Cosmetic and/or dermatological preparations for the purposes of the present invention may also comprise inorganic pigments which are customarily used in cosmetics to protect the skin against UV rays. These are oxides of titanium, zinc, iron, zirconium, silicon, manganese, aluminum, cerium and mixtures thereof, and also modifications in which the oxides are the active agents. Particular preference is given to pigments based on titanium dioxide. It is possible to use the amounts specified for the combinations above.

The cosmetic preparations according to the invention can comprise cosmetic auxiliaries, as are customarily used in such preparations, e.g. preservatives, bactericides, substances with a deodorizing action, antiperspirants, insect repellents, vitamins, antifoams, dyes, pigments with a coloring action, thickeners, softening substances, moisturizing and/or humectant substances, fats, oils, waxes or other customary constituents of a cosmetic formulation, such as alcohols, polyols, polymers, foam stabilizers, electrolytes, organic solvents or silicone derivatives.

Advantageously, preparations according to the invention can also comprise substances which absorb UV irradiation in the UVB region, where the total amount of filter substances is, for example, 0.1% by weight to 30% by weight, preferably 0.5 to 10% by weight, in particular 1.0 to 6.0% by weight, based on the total weight of the preparations in order to provide cosmetic preparations which protect the hair and/or the skin from the entire range of ultraviolet radiation. They can also serve as sunscreens for hair.

If the preparations according to the invention comprise UVB filter substances, these may be oil-soluble or water-soluble. Oil-soluble UVB filters advantageous according to the invention are e.g.:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicyclate, homomenthyl salicylate,
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate,
2,4,6-trianilino(p-carbo-2′-ethyl-1′-hexyloxy)-1,3,5-triazine.

Examples of advantageous water-soluble UVB filters are:
salts of 2-phenylbenzimidazole-5-sulfonic acid such as its sodium, potassium or its triethanolammonium salts, and the sulfonic acid itself;
sulfonic acid derivatives of benzophenones, preferably 2-hydroxy-4-methoxybenzophenone-5-sulfonic acid and its salts;
sulfonic acid derivatives of 3-benzylidenecamphor, such, for example, 4-(2-oxo-3-bornylidenemethyl)benzenesulfonic acid, 2-methyl-5-(2-oxo-3-bornylidenemethyl)sulfonic acid and its salts, and 1,4-di(2-oxo-10-sulfo-3-bornylidenemethyl)benzene and salts thereof (the corresponding 10-sulfato compounds, for example the corresponding sodium, potassium or triethanolammonium salts), also referred as benzene-1,4-di(2-oxo-3-bornylidenemethyl-10-sulfonic acid.

The list of specified UVB filters which can be used in combination with the active ingredient combinations according to the invention is not of course intended to be limiting.

It may also be advantageous to use UVA filters which are customarily present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. It is possible to use the amounts used for the UVB combination.

Cosmetic and dermatological preparations according to the invention advantageously also comprise inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. Particular preference is given to pigments based on TiO₂.

It is particularly advantageous for the purposes of the present invention, although not obligatory, if the inorganic pigments are present in hydrophobic form, i.e. have been superficially treated to repel water. This surface treatment can consist in providing the pigments with a thin hydrophobic layer by processes known per se.

Such a process consists, for example, in producing the hydrophobic surface layer by a reaction in accordance with
n TiO₂+m(RO)₃Si—R+→n TiO₂(surf.)
n and m are stoichiometric parameters to be used as desired, R and R′ are the desired organic radicals. Hydrophobicized pigments produced, for example, analogously to DE-A 33 14 742 are advantageous.

Advantageous TiO₂ pigments are available, for example, under the trade names MT 100 T from TAYCA, and also M 160 from Kemira, and T 805 from Degussa.

Preparations according to the invention may, especially when crystalline or microcrystalline solid bodies, for example inorganic micropigments, are to be incorporated into the preparations according to the invention, also comprise anionic, nonionic and/or amphoteric surfactants. Surfactants are amphiphilic substances which can dissolve organic, nonpolar substances in water.

The hydrophilic moieties of a surfactant molecule are mostly polar functional groups, for example —COO⁻—, —OSO₃²⁻, —SO₃⁻, whereas the hydrophobic moieties are usually nonpolar hydrocarbon radicals. Surfactants are generally classified according to the type and charge of the hydrophilic molecular moiety. In this connection, it is possible to differentiate between four groups:
anionic surfactants,
cationic surfactants,
amphoteric surfactants and
nonionic surfactants.

Anionic surfactants usually have, as functional groups, carboxylate, sulfate or sulfonate groups. In aqueous solution, they form negatively charged organic ions in an acidic or neutral medium. Cationic surfactants are characterized almost exclusively by the presence of a quaternary ammonium group. In aqueous solution, they form positively charged organic ions in an acidic or neutral medium. Amphoteric surfactants contain both anionic and cationic groups and accordingly in aqueous solution exhibit the behavior of anionic or cationic surfactants depending on the pH. In a strongly acidic medium, they have a positive charge, and in an alkali medium a negative charge. By contrast, in the neutral pH range, they are zwitterionic, as the example below is intended to illustrate:
RNH₂⁺CH₂CH₂COOH X⁻ (at pH=2) X⁻=any anion, e.g. Cl⁻
RNH₂⁺CH₂CH₂COO⁻ (at pH=7)
RNHCH₂CH₂COO⁻ B⁺ (at pH=12) B⁺=any cation, e.g. Na⁺

Typical nonionic surfactants are polyether chains. Nonionic surfactants do not form ions in aqueous medium.

A. Anionic Surfactants

Anionic surfactants which can be used advantageously are
acylamino acids (and salts thereof), such as
   1. acyl glutamates, for example sodium acyl glutamate, di-TEA-palmitoyl aspartate and sodium caprylic/capric glutamate,
   2. acylpeptides, for example palmitoyl-hydrolyzed milk protein, sodium cocoyl-hydrolyzed soya protein and sodium/potassium cocoyl-hydrolyzed collagen,
   3. sarcosinates, for example myristoyl sarcosine, TEA-lauroyl sarcosinate, sodium lauroyl sarcosinate and sodium cocoyl sarcosinate,
   4. taurates, for example sodium lauroyl taurate and sodium methyl cocoyl taurate,
   5. acyl lactylates, lauroyl lactylate, caproyl lactylate
   6. alaninates
carboxylic acids and derivatives, such as
   1. carboxylic acids, for example lauric acid, aluminum stearate, magnesium alkanolate and zinc undecylenate,
   2. ester carboxylic acids, for example calcium stearoyl lactylate, laureth-6 citrate and sodium PEG-4 lauramide carboxylate,
   3. ether carboxylic acids, for example sodium laureth-13 carboxylate and sodium PEG-6 cocamide carboxylate,
phosphoric esters and salts, such as, for example, DEA-oleth-10 phosphate and dilaureth-4 phosphate,
sulfonic acids and salts, such as
   1. acyl isethionates, e.g. isethionate,
   2. alkylarylsulfonates,
   3. alkylsulfonates, for example sodium cocomonoglyceride sulfate, sodium C₁₂₋₁₄-olefinsulfonate, sodium lauryl sulfoacetate and magnesium PEG-3 cocamide sulfate,
   4. sulfosuccinates, for example dioctyl sodium sulfosuccinate, disodium laureth sulfosuccinate, disodium lauryl sulfosuccinate and disodium undecyleneamido-MEA sulfosuccinate
and
sulfuric esters, such as
   1. alkyl ether sulfate, for example sodium, ammonium, magnesium, MIPA, TIPA laureth sulfate, sodium myreth sulfate and sodium C₁₂₋₁₃-parethsulfate,
   2. alkyl sulfates, for example sodium, ammonium and TEA lauryl sulfate.
      B. Cationic Surfactants

Cationic surfactants which can be used advantageously are
1. alkylamines,
2. alkylimidazoles,
3. ethoxylated amines and
4. quaternary surfactants
5. ester quats.

Quaternary surfactants comprise at least one N atom which is covalently bonded to 4 alkyl and/or aryl groups. Irrespective of the pH, this leads to a positive charge. Alkylbetaine, alkylamidopropylbetaine and alkylamidopropylhydroxysulfaine are advantageous. The cationic surfactants used according to the invention can also be preferably chosen from the group of quaternary ammonium compounds, in particular benzyltrialkylammonium chlorides or bromides, such as, for example, benzyldimethylstearylammonium chloride, and also alkyltrialkylammonium salts, for example for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl-or cetylpyrimidinium chloride, imidazoline derivatives and compounds with a cationic character, such as amine oxides, for example alkyl dimethylamine oxides or alkylaminoethyldimethylamine oxides. In particular, the use of cetyltrimethylammonium salts is advantageous.

C. Amphoteric Surfactants

Amphoteric surfactants which can be used advantageously are
1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,
2. N-alkylamino acids, for example aminopropylalkylglutamide, alkylaminopropionic acid, sodium alkylimidodipropionate and lauroamphocarboxyglycinate.
   D. Nonionic Surfactants

Nonionic surfactants which can be used advantageously are
1. alcohols,
2. alkanolamides, such as cocamides MEA/DEA/MIPA,
3. amine oxides, such as cocoamidopropylamine oxide,
4. esters which are formed by esterification of carboxylic acids with ethylene oxide, glycerol, sorbitan or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside
6. sucrose esters, sucrose ethers
7. polyglycerol esters, diglycerol esters, monoglycerol esters
8. methyl glucose esters, esters of hydroxy acids

Also advantageous is the use of a combination of anionic and/or amphoteric surfactants with one or more nonionic surfactants.

The surface-active substance may be present in the preparations according to the invention in a concentration between 1 and 95% by weight, based on the total weight of the preparations.

The lipid phase of the cosmetic or dermatological emulsions according to the invention can advantageously be chosen from the following group of substances:
mineral oils, mineral waxes
oils, such as triglycerides of capric or of caprylic acid, and also natural oils such as, for example, castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils, such as dimethylpolysiloxanes, diethylpolysiloxanes, diphenylpolysiloxanes and mixed forms thereof.

The oil phase of the emulsions of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12-18 carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Any mixtures of such oil and wax components can also be used advantageously for the purposes of the present invention. It may also in some instances be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can advantageously also have a content of cyclic or linear silicone oils, or consist entirely of such oils, although it is preferable to use an additional content of other oil phase components apart from the silicone oil or the silicone oils. Such silicones or silicone oils may be in the form of monomers, which are generally characterized by structural elements, as follows:

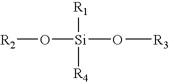

Linear silicones having two or more siloxyl units which are to be used advantageously according to the invention are generally characterized by structural elements, as follows: 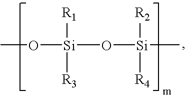
where the silicon atoms can be substituted by identical or different alkyl radicals and/or aryl radicals, which are shown here in general terms by the radicals R₁-R₄ (that is to say the number of different radicals is not necessarily limited to 4).
m can assume values from 2-200 000.

Cyclic silicones to be used advantageously according to the invention are generally characterized by structural elements, as follows 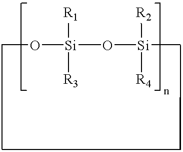
where the silicon atoms can be substituted by identical or different alkyl radicals and/or aryl radicals, which are shown here in general terms by the radicals R₁-R₄ (that is to say the number of different radicals is not necessarily limited to 4).
n can assume values from 3/2 to 20. Fractions for n take into consideration that uneven numbers of siloxyl groups may be present in the cycle.

Advantageously, cyclomethicone (e.g. decamethylcyclopentasiloxane) is used as the silicone oil to be used according to the invention. However, other silicone oils are also to be used advantageously for the purpose of the present invention, for example undecamethylcyclotrisiloxane, polydimethylsiloxane, poly(methylphenylsiloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated and/or polypropoxylated. These include, for example, polysiloxane-polyalkyl-polyether copolymers, such as cetyl-dimethicone copolyol, (cetyl-dimethicone copolyol (and) polyglyceryl-4-isostearate (and) hexyl laurate).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can advantageously be chosen from the group consisting of silicon dioxide and aluminum silicates.

Preparations according to the invention in the form of emulsions advantageously comprise, in particular, one or more hydrocolloids. These hydrocolloids can advantageously be chosen from the group of gums, polysaccharides, cellulose derivatives, phyllosilicates, polyacrylates and/or other polymers.

Preparations according to the invention in the form of hydrogels comprise one or more hydrocolloids. These hydrocolloids can advantageously be chosen from the abovementioned group.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar® HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorites.

In addition, silica gels can also be used advantageously.

The polyacrylates include, for example, Carbopol grades from Goodrich (Carbopol 980, 981, 1382, 5984, 2984, EDT 2001 or Pemulen TR2).

The polymers include, for example, polyacrylamides (Seppigel 305), polyvinyl alcohols, PVP, PVP/VA copolymers, polyglycols.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include
a) partial fatty acid esters and fatty acid esters of polyhydric alcohols and ethoxylated derivatives thereof (e.g. glyceryl monostearates, sorbitan stearates, glyceryl stearyl citrates, sucrose stearates)
b) ethoxylated fatty alcohols and fatty acids
c) ethoxylated fatty amines, fatty acid amides, fatty acid alkanolamides
d) alkylphenol polyglycol ethers (e.g. Triton X).

The anionic emulsifiers include
a) soaps (e.g. sodium stearate)
b) fatty alcohol sulfates
c) mono-, di-and trialkylphosphoric esters and ethoxylates thereof.

The cationic emulsifiers include
a) quaternary ammonium compounds with a long-chain aliphatic radical, e.g. distearyldimonium chloride.

The amphoteric emulsifiers include
a) alkylamininoalkanecarboxylic acids
b) betaines, sulfobetaines
c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:
fatty alcohol ethoxylates,
ethoxylated wool wax alcohols,
polyethylene glycol ethers of the general formula R—O—(—CH₂—CH₂—O—)ₙ—R′,
fatty acid ethoxylates of the general formula R—COO—(—CH₂—CH₂—O—)ₙ—H,
etherified fatty acid ethoxylates of the general formula R—COO—(—CH₂—CH₂—O—)ₙ—R′,
esterified fatty acid ethoxylates of the general formula R—COO—(—CH₂—CH₂—O—)ₙ—C(O)—R′,
polyethylene glycol glycerol fatty acid esters,
ethoxylated sorbitan esters,
cholesterol ethoxylates,
ethoxylated triglycerides,
alkyl ether carboxylic acids of the general formula R—O—(—CH₂—CH₂—O—)ₙ—CH₂—COOH and n are a number from 5 to 30,
polyoxyethylene sorbitol fatty acid esters,
alkyl ether sulfates of the general formula R—O—(—CH₂—CH₂—O—)ₙ—SO₃—H,
fatty alcohol propoxylates of the general formula R—O—(—CH₂—CH(CH₃)—O—)ₙ—H,
polypropylene glycol ethers of the general formula R—O—(—CH₂—CH(CH₃)—O—)ₙ—R′,
propoxylated wool wax alcohols,
etherified fatty acid propoxylates R—COO—(—CH₂—CH(CH₃)—O—)ₙ—R′,
esterified fatty acid propoxylates of the general formula R—COO—(—CH₂—CH(CH₃)—O—)ₙ—C(O)—R′,
fatty acid propoxylates of the general formula R—COO—(—CH₂—CH(CH₃)—O—)ₙ—H,
polypropylene glycol glycerol fatty acid esters,
propoxylated sorbitan esters,
cholesterol propoxylates,
propoxylated triglycerides,
alkyl ether carboxylic acids of the general formula R—O—(—CH₂—CH(CH₃)O—)ₙ—CH₂—COOH,
alkyl ether sulfates or the parent acids of these sulfates of the general formula R—O—(—CH₂—CH(CH₃)—O—)ₙ—SO₃—H,
fatty alcohol ethoxylates/propoxylates of the general formula R—O—Xₙ—Yₘ—H,
polypropylene glycol ethers of the general formula R—O—Xₙ—Yₘ—R′,
etherified fatty acid propoxylates of the general formula R—COO—Xₙ—Yₘ—R′,
fatty acid ethoxylates/propoxylates of the general formula R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly advantageously having having HLB values of 14.5-15.5, provided the O/W emulsifiers have saturated radicals R and R′. If the O/W emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to:
polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group:
polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, poly-ethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, and polyethylene glycol(20) oleate.

The ethoxylated alkyl ether carboxylic acid or salt thereof which can be used is advantageously sodium laureth-11 carboxylate.

Sodium laurethl-4 sulfate can be used advantageously as alkyl ether sulfate. An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

The examples below serve to illustrate the invention, but not limit it. The numerical data refer to percentage by weight, unless stated otherwise.

The examples below serve to illustrate the present invention.

### EXAMPLE 1

| | [g] |
|---|---|
| (A) Lipid phase | |
| Methyl palmitate | 77.10 |
| Hydroxyethylcellulose | 1.50 |
| Carbomer | 1.00 |
| Preservatives | 5.10 |
| (B) Water phase | |
| NaOH | 0.50 |
| Glycerol | 15.40 |
| Water | 914.10 |
| (C) Perfume phase | |
| Ethanol | 10.30 |
| Menthyl lactate | 1.00 |
| Perfume | 2.10 |

The constituents of phases (A) and (B) are in each case combined at about 80° C. and stirred until the phases appear homogeneous. Phases (A) and (B) are then combined and cooled to about 40° C. The perfume phase (C) is added to combined phases (A) and (B) at about 40° C., and the mixture is then cooled to room temperature.

### EXAMPLE 2

| | [g] |
|---|---|
| (A) Lipid phase | |
| Stearic acid | 81.50 |
| Methyl palmitate | 40.70 |
| (B) Water phase | |
| Triethanolamine | 8.146 |
| Glycerol | 20.366 |
| Water | 254.570 |
| (C) Perservative phase | |
| Phoxyethanol + methylparaben + | 8.146 |
| butylparaben + propylparaben + | |
| isobutylparaben | |

The constituents of phases (A) and (B) are in each case combined at about 70-75° C. and stirred until the phases appear homogeneous. Phases (A) and (B) are then combined and cooled to about 60° C., where they are emulsified by vigorous stirring. The preservative phase (C) is added to the combined phases (A) and (B) at about 50° C., after which the entire mixture is cooled to room temperature with continued stirring.

### EXAMPLE 3

| | [g] |
|---|---|
| (A) Lipid phase | |
| Stearic acid | 203.383 |
| Methyl palmitate | 101.691 |
| Dimethicone | 10.169 |
| Cyclomethicone | 10.169 |
| (B) Water phase | |
| Triethanolamine | 18.304 |
| Glycerol | 101.691 |
| Water | 526.252 |
| (C) Perservative phase | |
| Ethanol | 30.507 |
| Menthyl lactate | 7.627 |
| Perfume | 2.034N |
| Phenoxyethanol + methylparaben + | 5.085 |
| butylparaben + propylparaben + | |
| isobutylparaben | |

The constituents of phases (A) and (B) are in each case combined at about 70-75° C. and stirred until the phases appear homogeneous. Phases (A) and (B) are then combined and cooled to about 60° C., where they are emulsified and 10 homogenized by vigorous stirring. Preservative phase (C) is added to the combined phases (A) and (B) at about 55° C., after which the entire mixture is cooled to room temperature with continued stirring.

### EXAMPLE 4

| | [g] |
|---|---|
| (A) Lipid phase | |
| Stearic acid | 203.383 |
| Methyl palmitate | 101.880 |
| Dimethicone | 10.188 |
| Cyclomethicone | 10.188 |
| Menthyl lactate | 7.641 |
| Phenoxyethanol + methylparaben + | 5.094 |
| butylparaben + propylparaben + | |
| isobutylparaben | |
| (B) Water phase | |
| Triethanolamine | 18.338 |
| Glycerol | 50.940 |
| Water | 608.735 |
| (C) Perfume phase | |
| Perfume | 2.038N |

The constituents of phases (A) and (B) are in each case combined at about 70-75° C. and stirred until the phases appear homogeneous. The phases (A) and (B) are then combined and cooled to about 60° C., where they are emulsified and homogenized by vigorous stirring. The perfume phase (C) is added to the combined phases (A) and (B) at about 55° C., after which the entire mixture is cooled to room temperature with continued stirring.

1. A cooling cosmetic or medicinal topical preparation comprising one or more substances having a melting point or melting interval between 28° C. and 40° C. and a positive melting enthalpy, wherein the one or more substances are present in the preparation in the solid state.

2. The preparation as claimed in claim 1, wherein said one or more substances are present in an concentration of 0.5-50% by weight, based on the total weight of the preparation.

3. The preparation as claimed in claim 1, wherein said one or more substances are present in an concentration of 1-20% by weight, based on the total weight of the preparation.

4. The preparation as claimed in claim 1, wherein said one or more substances are selected from the group consisting of lipophilic substances.

5. The preparation as claimed in claim 4, wherein the preparation comprises a markedly hydrophilic cosmetic or dermatological base and said one or more substances are preferably present in a finely particulate suspension in said base.

6. The preparation as claimed in claim 5, wherein the preparation is an aqueous gel formulation.

7. The preparation as claimed in claim 1, wherein said one or more substances are selected from the group consisting of esters of C₁₄-C₁₈ fatty acids and low molecular weight (C₁-C₅) alcohols.

8. The preparation as claimed in claim 1, wherein said one or more substances are selected from the group consisting of C₈-C₂₂ fatty alcohols.

9. The preparation as claimed in claim 1, wherein said one or more substances are selected from the group consisting of branched and unbranched fatty acids.

10. The preparation as claimed in claim 1, wherein said one or more substances are selected from the group consisting of C₁₆-C₂₂ hydrocarbons.

11. The preparation as claimed in claim 1, wherein said one or more substances include methyl palmitate.

12. A method for producing a cooling action on the skin, comprising the step of applying to the skin a cosmetic or medicinal topical preparation comprising one or more substances having a melting point or melting interval between 28° C. and 40° C. and a positive melting enthalpy, wherein the one or more substances are present in the preparation in the solid state.

13. The method as claimed in claim 12, wherein said one or more substances are present in an concentration of 1-20% by weight, based on the total weight of the preparation.

14. The method as claimed in claim 12, wherein said one or more substances are selected from the group consisting of lipophilic substances.

15. The method as claimed in claim 14, wherein the preparation comprises a markedly hydrophilic cosmetic or dermatological base and said one or more substances are preferably present in a finely particulate suspension in said base.

16. The method as claimed in claim 15, wherein the preparation is an aqueous gel formulation.

17. The method as claimed in claim 12, wherein said one or more substances are selected from the group consisting of esters of C₁₄-C₁₈ fatty acids and low molecular weight (C₁-C₅) alcohols.

18. The method as claimed in claim 12, wherein said one or more substances are selected from the group consisting of C₈-C₂₂ fatty alcohols.

19. The method as claimed in claim 12, wherein said one or more substances are selected from the group consisting of branched and unbranched fatty acids.

20. The method as claimed in claim 12, wherein said one or more substances are selected from the group consisting of C₁₆-C₂₂ hydrocarbons.

21. The method as claimed in claim 12, wherein said one or more substances include methyl palmitate.
